(19) United States
(12) Patent Application Publication
(10) Pub. No.: US 2017/0240856 A1
(43) Pub. Date: Aug. 24, 2017

(54) Placenta-derived potential cells and preparing method thereof

(71) Applicant: Shanghai STEMsan Biotechnology Co.,Ltd., Shanghai (CN)

(72) Inventors: Jigang Zhang, Shanghai (CN); Haochuan Wang, Shanghai (CN)

(73) Assignee: Shanghai STEMsan Biotechnology Co.,Ltd.

(21) Appl. No.: 15/591,067

(22) Filed: May 9, 2017

(30) Foreign Application Priority Data
May 9, 2016 (CN) 201610300333.5

(51) Int. Cl. C12N 5/073

(52) U.S. Cl. CPC C12N 5/0605; C12Y 304/24; C12Y 304/24003; C12N 2506/025; C12N 2509/00; C12N 2500/84; C12N 2501/40; C12N 2500/60; C12N 2500/38; C12N 2500/34; C12N 2500/30; C12N 2500/32

(57) ABSTRACT

A method for culturing placental potential cell is provided, comprising steps of: (1) obtaining placental cells and/or tissue under aseptic condition; (2) inoculating the placental cells and/or the tissue in a culture medium for culturing, adding cell growth regulators to the culture medium, in such a manner that the placental potential cells grows to make the placental cells and/or the tissue into a proliferative state; (3) culturing the placental potential cells to make the placental potential cells proliferate continuously into cells with characteristics of stem cells. The present invention not only finds the source of human tissues, organs and the continuation of their function, i.e., regenerative potential cells; but also finds a medical and health longevity method, but also finds out the life materials to maintain and support the potential cells, so as to replace drugs with the living material.

### CROSS REFERENCE OF RELATED APPLICATION

The present application claims priority under 35 U.S.C. 119(a-d) to CN 201610300333.5, filed May 9, 2016.

### BACKGROUND OF THE PRESENT INVENTION

Field of Invention

The present invention relates to a cell with potential, and more particularly to a cell with potential which is derived from animal placenta and a culturing method thereof.

Description of Related Arts

From the end of the 20^{th}century, the researches of life sciences have found that stem cells can be cultured in vitro and developed into cells or tissues having specificity of various organs of human body. Thus, culturing organs in vitro is expected in the near future to perform organs auto-transplantation for treating diseases. Alternatively, stem cells are injected into a part of the human body, the stem cells perform a function of an organ in partial, and finally the object of treating diseases is achieved. However, in the culturing process of stem cells, in-vitro stem cells culture technology is limited by the rapid aging and self-differentiation of stem cells, i.e. lose stemness.

In the conventional arts, cell culture refers to the use of available technology to provide an in vitro environment in which cells grows, proliferates, maintains structure and function under a sterile condition with appropriate temperature, pH and certain nutrients. The cell culture includes the growth of a single cell or tissue.

A non-toxic and sterile environment is the basic requirement for cell culture and a primary condition to ensure cell survival. Compared with the in-vivo condition, when cells are disposed in vitro for culturing, the cells lose the defense capability against microorganism and toxic substance. The pollution and accumulation of metabolic substances may lead to cells deteriorate and die. Thus, during the culturing process, keeping the cells in an environment without any pollution and removing the metabolites in time is essential for maintaining survival of the cells.

The suitable temperature of the cell culture is another condition for maintaining the vigorous growth of the cultured cells. The standard temperature of human cell culture is at a range of 36.5° C.±0.5° C., and if the temperature range is deviated, the normal metabolism of cells will be affected, or the cells even died.

Gas is also one of the necessary conditions for the survival of human cell culture, and the gases required mainly include oxygen and carbon dioxide, wherein oxygen is involved in tricarboxylic acid cycle to produce energy required for cell growth. During the culture, the cells are often disposed in a mixed gas environment with 95% air plus 5% carbon dioxide.

Carbon dioxide is both a cellular metabolism and a required component for cell growth. The main role of carbon dioxide in cell culture lies in maintaining the pH of the culture medium. Most cells have a suitable pH at a range of 7.2-7.4, and deviating from this range will have a detrimental effect on cell culture. The most commonly used method for regulating pH of the cell culture medium is by adding NaHCO₃, that's because NaHCO₃ is capable of providing CO₂. However, CO₂ is easy to escape, so adding NaHCO₃ is most suitable for closed culture. N-2-hydroxyethylpiperazine-N-ethane-sulphonic acid (HEPES) is non-toxic and capable of playing a buffering role, and has characteristic of preventing fast variation of pH value, and thus HEPES is applied in the technique of opening cell culture. The greatest advantage of HEPES is capable of maintaining a constant pH value during open culturing.

Furthermore, the cell culture medium is not only a basic material for providing the cell culture with nutriment for growth and proliferation, but also the living environment for culturing the cell to grow and proliferate. The culture medium has various types, and can be classified into synthetic medium and natural medium according to their source.

(1) Synthetic Medium

Synthetic Medium is prepared strictly according to the types and amount of the substances required by the cells, which contains carbohydrates, amino acids, lipids, inorganic salts, vitamins, trace elements and cell growth factors. When the types of the materials are used alone, the cells may survive, but are not capable of growing well.

(2) Natural Medium

The most commonly used natural medium is serum, wherein calf serum is the most common type. Since the serum contains a variety of cell growth factors, adhesion factors and a variety of active substances, when combined with synthetic media, the serum is capable of making the cells grow well, wherein a commonly used concentration is at a range of 5-20%.

The cultured cells can be classified into attached cells and suspended cells; wherein the attached growth cells are capable of growing by attaching the surface of the supporter. The common attached growth cells include fibroblasts, epithelial cells and migratory cells. The suspended growth cells grow in suspension in culture.

1. Fibroblasts

In the cell culture, cells which have morphology similar to fibroblasts can all be called fibroblasts. The cells are grown on a surface of the supporter in a spindle or irregular triangular shape; wherein an ovoid nucleus is in the center of the cells, protrusions with different length are extended out of the cytoplasm. Except real fibroblasts, the tissue cells origin from mesenchymal of mesoderm often show similar morphology and characteristics of growth.

2. Epithelial Cells

These kinds of cells grow on the culture vessel support and have characteristic of flat irregular polygonal. Circular cells are in the center of the cells, and the cells are closely linked and grown in a monolayer membrane shape. Cells derived from tissues of entoderm or ectoderm such as skin, epidermal derivatives, digestive epithelial cells all belong to epithelial cell type.

3. Migratory Cells

This kind of cells are grown by distributed on the supporter, and generally not linked into flakes. Pseudopods or protrusions are often stretched out of the cytoplasm, with active walking or deformed movements, wherein the speed is fast and irregular. This kind of cells is not very stable, and sometimes it is difficult to distinguish them from other kinds of cells. Under a certain condition, due to increased cell density, the cells are linked into flakes, the cells show a shape similar to the polygonal or fibroblast morphology, which is common in the early culture of amniotic fluid cell.

Morphological Analysis of the Cultured Cells

The shapes of cultured cells vary with the shape of the supporter attached. The most common type is the cells attached on a flat supporter. In the general light microscope, the cells are homogeneous and transparent, and the structure is not obvious. During the growing stage of the cells, often 1-2 nucleoli will increase the cell contour under a poor cell functional state, and the contrast increases. If the particles and vacuoles and etc. emerge in the cytoplasm, poor cell metabolism is indicated.

### SUMMARY OF THE PRESENT INVENTION

An object of the present invention is to provide a potential cell derived from a placenta, named a placental potential cell, which is a cell has a proliferation potential of a stem cell and exists in tissue with common cell morphology.

Another object of the present invention is to provide a culturing method of the potential cell derived from the placenta.

Another object of the present invention is to provide a cell growth regulator for in vitro cell culture, and particularly for the cell culture of human.

Another object of the present invention is to provide a culture medium containing the cell growth regulator of the present invention.

Another object of the present invention is to provide an application of the placental potential cell cultured in vitro by the method of the present invention.

Accordingly, in order to achieve the object mentioned above, the present invention provides a human placental potential cell capable of proliferating continuously in vitro, wherein the human placental potential cell has proliferation potential of stem cells and is presented in tissue with morphology of a normal tissue cell; wherein a culturing method of the placental potential cell comprises steps of:
(1) obtaining placental cells and/or tissue under aseptic condition;
(2) seeding the placental cells and/or the tissue in a culture medium for culturing, adding cell growth regulators to the culture medium, in such a manner that the placental potential cells grow to make the placental cells and/or the tissue into a proliferative state;
(3) culturing the placental potential cells to make the placental potential cells proliferate continuously into cells with characteristics of stem cells.

Preferably, the cells which are proliferated form various types of tissue under in-vitro induction conditions.

Preferably, the inoculating the placental cells and/or the tissue in the culture medium for culturing of the step (2) mentioned above comprises steps of:

(2-A) washing the placental cells and/or the tissue with phosphate buffer (PBS) containing at least two types of antibiotics comprising penicillin and streptomycin for at least 2 times, 30 seconds-3 minutes for each time;

(2-B) washing the placental cells and/or the tissue to a complete medium containing at least two types of antibiotics comprising the penicillin and the streptomycin for at least 2 times, 30 seconds-3 minutes for each time;

(2-C) cutting the placental cells and/or the tissue into explants with a size at a range of 1-5 mm³;

(2-D) placing the organ type explants into a center of the culture microplates of the culture plate in 1-5 mm interval, slightly pressing each of the organ type explants to make each of the organ type explants tightly cling to a surface of the culture plate;

(2-E) along each periphery of each of the culture microplates, adding 0.1-1.0 ml the complete medium to each of the culture microplates, preventing the complete medium from contacting with the organ type explants;

(2-F) placing the culture plate into an incubator at 37° C. with 1-10 % CO₂ to pre-culture for 0.5-2 hours; and

(2-G) gently adding the complete medium to each of the microplates, preventing the explants from floating, and adding the cell growth regulator.

Preferably, the inoculating the placental cells and/or the tissue in the culture medium for culturing of the step (2) mentioned above comprises steps of:

(2-a) in order to separate the placental tissue into individual cells, firstly putting the placental tissue into 4° C. pre-cooled phosphate buffered saline (PBS) containing at least two types of antibiotics comprising penicillin and streptomycin, washing for at least 3 times, 30 seconds to 3 minutes for each time; then cutting the placental tissue into minimal organ type explants with a size at a range of 1-5 mm³, washing for two times with cooled PBS containing at least two types of antibiotics; then sending the placental tissue into 0.1-0.5% neutral protease solution or 0.5-2% collagenase solution which is prepared by sterile PBS for digestion, wherein conditions are 36.5-37° C. constant temperature oscillation for 0.5-3 hours;

(2-b) gently mixing the explants by pipetting; or pouring the explants digested with 0.1-0.5% dispase solution or 0.5-2% collagenase solution (in sterile PBS) onto a stainless steel sieve, grinding the explants with a syringe until the explants are completely discrete and become single cells; keeping mixture containing the single cells and digestive enzyme which is known to skills in the art for 2-8 minutes, discarding precipitated chunks and non-digestible connective tissue; transferring supernatant containing plenty of the single cells and the digestive enzyme to another centrifugal tube; filtering the supernatant once with a stainless steel filter, so as to obtain a mixture of the digestive enzyme and the single cell;

(2-c) centrifuging for 3-20 minutes with 1000-3000 r/min at 4° C., discarding supernatant containing the digestive enzyme, spinning the single cells by a vortex with PBS which is pre-cooled at 0.5-6° C.; then adding PBS pre-cooled at 0.5-6° C.; and mixing well;

(2-d) centrifuging for 3-20 minutes at 0.5-6° C. with a rate 1000-3000 r/min;

discarding supernatant, adding cool PBS, oscillating with a vortex to spin the single cells; then adding PBS pre-cooled at 0.5-6° C.; mixing well; and counting the single cells;

(2-e) centrifuging for 3-20 minutes at 0.5-6° C. with a rate 1000-3000 r/min; discarding supernatant, adding alpha MEM culture solution containing 10-20% fetal bovine serum; oscillating by vortex to spin up the single cells, then adding the alpha MEM culture solution containing 10-20% fetal bovine serum to regulate the cell concentration at 1×10⁵/ml, mixing well to obtain a quantitative cell suspension;

(2-f) adding the cell suspension to a plastic porous plate which is a 96-well plate, 24-well plate, 12-well plate or a 6-well plate; wherein adding 2000 to each well of the 96-well plate; adding 1 ml to each well of the 24-well plate; adding 2 mL to each well of the 12-well plate and adding 4 mL to each well of the 6-well plate;

(2-g) sending the plastic porous plate to a cell incubator containing 5% CO₂ at 36.5-37° C. for 12-48 hours until all the cells are adherent.

According to a further preferred embodiment of the present invention, the inoculating the placental cells and/or the tissue in the culture medium for culturing of the step (2) mentioned above comprises steps of:

(2-A′) continuously washing the placental cells and/or the tissue with phosphate buffer (PBS) containing at least two types of antibiotics comprising penicillin and streptomycin for at least 2 times, 1 minute for each time;

(2-B′) sending the placental cells and/or the tissue to a complete medium containing at least two types of antibiotics comprising the penicillin and the streptomycin for at least 2 times, 1 minute for each time;

(2-C′) cutting the placental cells and/or the tissue into minimal organ type explants with a size at 2 mm×2 mm×2 mm;

(2-D′) sending the organ type explants into a center of the culture microplates of the culture plate in 2 mm interval, slightly pressing each of the organ type explants to make each of the organ type explants tightly cling to a surface of the culture plate;

(2-E′) along each periphery of each of the culture microplates, adding 0.5 ml the complete medium to each of the culture microplates, preventing the complete medium from contacting with the organ type explants;

(2-F′) sending the culture plate into an incubator at 37° C. with 5% CO₂ to pre-culture for 1 hour; and

(2-G′) gently adding 2 ml the complete medium to each of the microplates, preventing the organ type from floating, and adding the cell growth regulator.

Preferably, the inoculating the placental cells and/or the tissue in the culture medium for culturing of the step (2) mentioned above comprises steps of:

(2-a′) in order to separate the placental tissue into individual cells, firstly putting the placental tissue into 4° C pre-cooled phosphate buffered saline (PBS) containing at least two types of antibiotics comprising penicillin and streptomycin, washing for at least 3 times, 30 seconds to 3 minutes for each time; then cutting the placental tissue into minimal organ type explants with a size at 2 mm³, washing for two times with cooled PBS containing at least two types of antibiotics; then sending the placental tissue into 0.25% neutral protease solution or 1% collagenase solution which is prepared by a bacterial PBS for digestion, wherein conditions are 37° C. constant temperature oscillation for 1 hours;

(2-b′) repeatedly blowing and beating the minimal organ type explants with a sampler or a straw; or pouring the minimal organ type explants and the 0.1-0.5% neutral protease solution or 0.5-2% collagenase solution which is prepared by a bacterial PBS for digestion onto a stainless steel screen, grinding the organ type explants with a syringe until the organ type explants are completely discrete and become single cells; standing mixture containing the single cells and digestive enzyme which is known to skills in the art for 5 minutes, discarding precipitated chunks and non-digestible connective tissue; transferring supernatant containing plenty of the single cells and the digestive enzyme to another centrifugal tube; filtering the supernatant once with a stainless steel filter, so as to obtain a mixture of the digestive enzyme and the single cell;

(2-c′) centrifuging for 5 minutes with a rate 1500 r/min at 4° C., discarding supernatant containing the digestive enzyme, spinning the single cells by a vortex with cooled PBS; then adding cooled PBS; and mixing well;

(2-d′) centrifuging for 5 minutes at 4° C. with a rate 1500 r/min; discarding supernatant, adding cool PBS, oscillating with a vortex to spin the single cells; then adding cooled PBS; mixing well; and counting the single cells;

(2-e′) centrifuging for 5 minutes at 4° C. with a rate 1500 r/min; discarding supernatant, adding alpha MEM culture solution containing 15% fetal bovine serum; oscillating by vortex to spin up the single cells, then adding the alpha MEM culture solution containing 15% fetal bovine serum to regulate the cell concentration at 1×10⁵/ml, mixing well to obtain a quantitative cell suspension;

(2-f′) adding the cell suspension to a plastic porous plate which is a 96-well plate, 24-well plate, 12-well plate or a 6-well plate; wherein adding 2000 to each well of the 96-well plate; adding 1 ml to each well of the 24-well plate; adding 2 mL to each well of the 12-well plate and adding 4 mL to each well of the 6-well plate;

(2-g′) sending the plastic porous plate to a cell incubator containing 5% CO₂ at 37° C. for 24 hours until all the cells are adherent.

Preferably, a source of the placenta comprises: a spontaneous abortion human placenta, a mice placenta, a hamster placenta, a pig placenta or a cow placenta.

Preferably, components of the medium comprise:

1) 75-85% by mass fraction of amino acids selected from one or more member from the group consisting of: arginine, cystine, glutamine, histidine, isoleucine, leucine, lysine, methionine, phenylalanine, threonine, tryptophan and valine;

2) 3-5% by mass fraction of vitamins selected from one or more member from the group consisting of: biotin, choline, folic acid, nicotine, pantothenic acid, vitamin B6, vitamin B1 and riboflavin;

3) 0.6-1.2% by mass fraction of salt selected from one or more member from the group consisting of: NaCl, KCl, NaHPO₄, NaHCO₃, CaCl₂ and MgCl₂;

4) 8-12% by mass fraction of salt selected from one or more member from the group consisting of: insulin and transferrin;

5) 1.8-2.4% by mass fraction of glucose, 1.2-1.8% by mass fraction of penicillin and 1.2-1.8% by mass fraction of streptomycin.

Preferably, the cell growth regulator is selected from the group consisting of: resveratrol, amino acids, fetal bovine serum and human serum.

A placental potential cell cultured by the method mentioned above.

Preferably, a content of the resveratrol in the medium is 10-60 ng/mL.

The present invention provides an application of the placental potential cell.

The culture result can be observed by an inverted microscope, and the results are observed dynamically by different method such as visual field, phase difference, differential dry radiation, Huffman and fluorescence. Cell counting; biological dye or fluorescent dye staining; cell proliferation detection; cell histochemical detection; immune cell histochemistry and so on are carried out in the present invention.

By adding the cell culture regulator of the present invention, the culture medium of the present invention is capable of continuously culturing various cells or tissues of the human. Under a suitable condition, the culture time can be up to 90-180 days or even longer.

In addition, in the conventional art, it is generally attempted to directly train stem cells in vitro to enable them to differentiate into the desired somatic cells. The object of the present invention is that directly by the method provided by the present invention, the stem cells of human beings are capable of obtaining normal cells and tissues with corresponding function of various cells and tissues on the medium of the present invention.

The cells and tissues obtained in vitro by the present invention can serve as an experimental model to carry out safety and efficiency experiments on drugs; to screen living substances having maintenance and regeneration function on tissues and cells; and screen therapeutic substances for the diseases of the tissue itself.

Thus, the present invention cultures cells in vitro having a relatively physiological ability to proliferate, and then culture the continuously proliferating cells to clone and replicate, so as to form functional tissues.

Protein components extracted from the potential cells cultured by the culturing method of the present invention can be utilized to repair the patient and the damaged tissues, and to induce apoptosis of the cancer cells.

It can be seen from above that the objects of the present invention is as follows:
1. researching and studying research model of in vitro cells and tissues;
2. searching materials that maintains and support organizations and organs;
3. by researching cells to protect the health condition of human beings or animals, so as to achieve objects of preventing and treating diseases and keeping healthy and longevity;
4. utilizing the living materials of the cells and tissues obtained to repair damaged cells and inhibit cancer cells.

According to the results of the present invention, beneficial effects of the present invention are as follows.

Human placental potential cells can be continuously proliferated to supplement cells or tissues that have been apoptotic, degenerated, and damaged, so as to maintain their tissue structure and function. In vivo, the human placental potential cells participate in the formation of the structure and function of the tissues and organs in a manner of common cells, so as to combine with tissues formed by the proliferated stem cells to form organs. When cells of the organs that are apoptosis, degradation or damage necrosis, these potential cells start their own proliferation function to regenerate new cells to replicate, so as to timely replenish the cells, tissues and functional vacancies in the organ, and thus restore the structure and function of the organs, to ensure normal function of organ tissue.

The present invention is capable of achieving situ replication of the skin and gastrointestinal mucosa.

Through the present invention, placenta potential cells are capable of developing into tissues of cartilage, bone marrow, fat, endothelium and nerve, and finally all tissue and organs are expected to form.

In other words, the scientific and medical and economic value of the present invention is of great significance. On the one hand, the present invention not only finds the source of human tissues, organs and the continuation of their function, i.e., regenerative potential cells; but also finds a medical and health longevity method, but also finds out the life materials to maintain and support the potential cells, so as to replace drugs with the living material.

On the other hand, through the study of the effect of the protein extract of the invention on the repair of tissues and organs, the present invention obtains the method of protecting the physiological function of human tissues and organs, realizing health and longevity thereof, and preventing the occurrence of tissue and organ diseases.

In addition, through the replication of organization the present invention is capable of repairing tissue organs in physiological to maintain the balance of life organs; treating difficult diseases; and making the patients out of medical pain and injury.

The cell growth regulator of the present invention can also be used for the culture of other cells or tissues.

Terms in the Present Invention

In order to facilitate the elaboration of the research process of the present invention, the terminology used in the present invention is first determined to distinguish the same as the conventional concept, and the specific terms of the invention are as follows:

Tissue Organ:

In the prior art, the concept of “organ” in human tissue and organ in the international community is a functional organization unit. Zhang Zhaoyou's “human anatomy” (1996 People's Health Publishing House) that “different types of cells, with a cell as the theme, respectively, constitute a different organization, a variety of organizational organs.

Wang Jingmei “Human Anatomy” (1994 People's Health Publishing House) that: “many similar shape, similar function of cells by the intercellular stroma together, the formation of the structure into the organization; several different organizations in accordance with certain laws Distribution, composition of a certain form and function of the structure, as organs.

The term “tissue” as used in the present invention means that the regenerated in situ and in vitro “tissue” is formed by “placental potential cells” and then forms a functional unit.

Placental Potential Cells:

The inventors of the present invention have found that cells having regenerative capacity in the human placenta are named placental potential cells which are cells that have the potential for stem cell proliferation and which can differentiate into tissue under specific conditions. Also known as potential cells (Potential Cell). In the histology, the potential cells can be into other tissue cells, and even can become the original cells. Therefore, when the organ disease necrosis, placental potential cells can be timely regeneration of one or more cells to compensate for vacancies, so that there will be no human organ necrosis phenomenon.

Therefore, the potential cells in the placenta can be continuously proliferated to supplement the apoptosis, degeneration and damage of necrotic tissue cells in order to maintain its organizational structure and function to achieve. These cells are associated with normal tissue cells, not like stem cells like proliferation or differentiation, so it is named “potential cells.” When the organization of organ apoptosis, degradation, damage necrosis, these potential cells to start their own proliferation function, regeneration of new cells, replenish the organs in time cells, tissue vacancies, timely restoration of organ structure and function, to protect the organ tissue Function of the normal play. The body of all organs of the repair function to play a normal, the body will be able to maintain the balance of life as a whole to achieve its health and longevity; if an organ or tissue of this function cannot play or low, an organ or tissue will produce disease.

These and other objectives, features, and advantages of the present invention will become apparent from the following detailed description, the accompanying drawings, and the appended claims.

### BRIEF DESCRIPTION OF THE DRAWINGS

FIG. 1 is a sketch view showing manufacturing process of potential cells, wherein the potential cells are potential cells with proliferating ability invented by the present invention.

FIG. 2 is a first microscopic diagram after cultured by the experimental group, wherein the cells are capable of performing sustained proliferation and division and differentiating into various tissues.

FIG. 3 is a second microscopic diagram after cultured by the experimental group, wherein the cells are capable of performing sustained proliferation and division and differentiating into various tissues.

FIG. 4 is a sketch view of two pairs of adherent placental potential cells by adding fat inducing agent, wherein the two pairs of adherent placental potential cells are fixed after culturing for 21 days, and then stained with oil red, wherein oil red is dissolved in lipids and insoluble in water, and is commonly used for lipid staining; wherein lipid droplets are found in the differentiated cells in the FIG. 4. (Magnified multiple 10×)

FIG. 5 is a microscopic image of the osteogenic tissue of the experimental group in the second preferred embodiment; wherein after an identical batch of cells adhered, adding osteogenic inducer, culturing for 21 days and fixing, staining with alizarin red, wherein alizarin red is capable of chelating with calcium ions to form a complex. (Magnified multiple 10×).

FIG. 6 is an image of stained cartilage tissue in the preferred embodiment 2, wherein the cartilage tissue is formed by adding inducer to cells, culturing for 21 days and staining.

FIG. 7A-I is a sketch view of cells cultured for 30 days according to the preferred embodiment 3, wherein cell surface markers are determined by flow cytometry. Referring to FIG. 7A-I, the cells in experimental groups express CD90, CD73, CD105, and fail to express CD31, CD45, CD14, CD34, CD9 and HLA-DR.

### DETAILED DESCRIPTION OF THE PREFERRED EMBODIMENT

In order to facilitate the understanding of those skilled in the art, the technical solution adopted by the present invention is to obtain human in situ organ tissue cells; culture the tissue cells in vitro, promote placental potential cell cloning to proliferate functional tissue; extract proteins wherein the extracted proteins is capable of promoting cell growth and tissue repair.

### Embodiment 1

### Culturing Human Placental Cells in Vitro

Human placenta is obtained and then performing steps as follows:

continuously washing the placental tissue with 4° C. pre-cooled phosphate buffer (PBS) containing penicillin and streptomycin for at least 3 times;

cutting the big block of placental tissue into minimal organ type explants with a size at 2 mm×2 mm×2 mm; and then washing with the 4° C. pre-cooled phosphate buffer (PBS) containing penicillin and streptomycin for at least 2 times;

sending the placental tissue into 0.25% neutral protease solution or 1% collagenase solution which is prepared by a bacterial PBS for digestion, wherein conditions are 37° C. constant temperature oscillation for 1 hours; and then performing steps as follows:

repeatedly blowing and beating the minimal organ type explants with a sampler or a straw; or pouring the minimal organ type explants and the neutral protease solution or collagenase solution which is prepared by a bacterial PBS for digestion onto a stainless steel screen, grinding the organ type explants with a syringe until the organ type explants are completely discrete and become single cells;

standing mixture containing the single cells and digestive enzyme which is known to skills in the art, such as such as neutral protease and collagenase, for 5 minutes, discarding precipitated chunks and non-digestible connective tissue; transferring supernatant containing plenty of the single cells and the digestive enzyme to another centrifugal tube; filtering the supernatant once with a stainless steel filter, so as to obtain a digestive mixture;

centrifuging for 5 minutes with a rate 1500 r/min at 4° C., discarding supernatant containing the digestive enzyme, spinning the single cells by a vortex with cooled PBS; then adding cooled PBS; and mixing well;

centrifuging for 5 minutes at 4° C. with a rate 1500 r/min; discarding supernatant, adding cool PBS, oscillating with a vortex to spin the single cells; then adding cooled PBS; mixing well; and counting the single cells;

centrifuging for 5 minutes at 4° C. with a rate 1500 r/min; discarding supernatant, adding alpha MEM culture solution containing 15% fetal bovine serum; oscillating by vortex to spin up the single cells, then adding the alpha MEM culture solution containing 15% fetal bovine serum to regulate the cell concentration at 1×10⁵/ml, mixing well to obtain a quantitative cell suspension;

adding the cell suspension to a plastic porous plate which is a 96-well plate, 24-well plate, 12-well plate or a 6-well plate; wherein adding 2000 to each well of the 96-well plate; adding 1 ml to each well of the 24-well plate; adding 2 mL to each well of the 12-well plate and adding 4 mL to each well of the 6-well plate;

sending the plastic porous plate to a cell incubator containing 5% CO₂ at 37° C. for 24 hours until all the cells are adherent.

After culturing for 24 hours, the adherent cells are observed growing normally under microscope. The culture wells were divided into two groups. One group was the experimental group and the other group was the control group. In the experimental group, alpha MEM medium and resveratrol containing 15% fetal bovine serum are added. The selection of cell culture media is well known to those skilled in the art. The control group did not include the substance of the present invention. The cell culture regulator was added in an amount of 10 ng-60 ng/ml in the medium.

According to the procedures required, replace the culture medium. A liquid replacement method is removing half of the old culture medium, and adding the same amount of fresh alpha MEM culture medium with 15% fetal bovine serum. Then the culture solution is changed in each 3 days, and combination of regular observation and observation at any time are adopted.

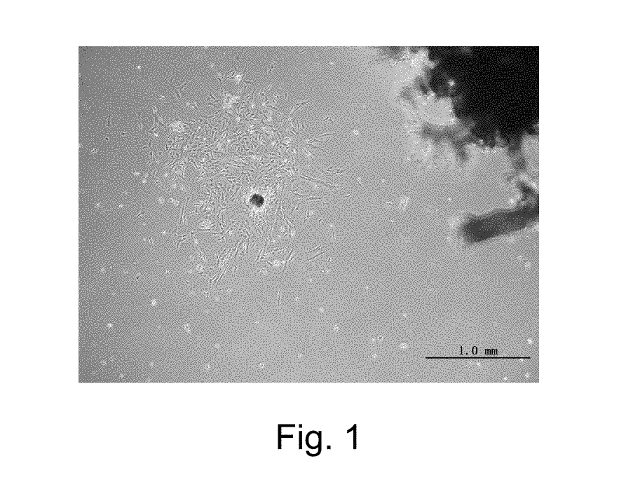

After 25 days of culture, as shown in FIG. 1, human placental cells are in a single form in the culture medium, at this moment the cells survive in the culture medium, and the life is active, and some cells are in split phase. Some cells in these cells have the ability of continuously proliferating, we call placental potential cells; some cells do not have the ability of continuously proliferating.

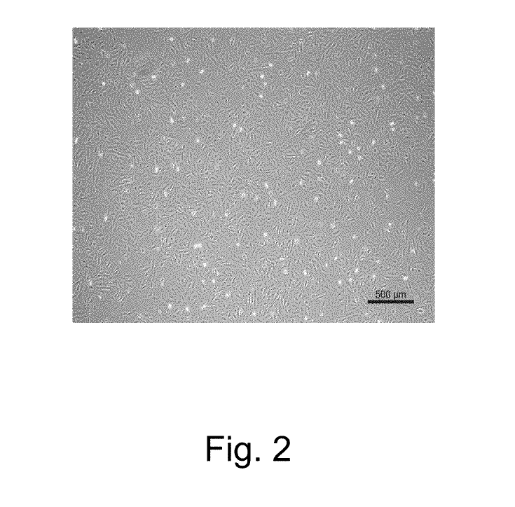

Referring to FIG. 2, it can be seen that the larger cells that are dividing are cells that are replicating the tissues and organs of the placental potential cells; the smaller undifferentiated cells are the original non-proliferating and newly produced placental potential cells; these cells proliferate continuously in culture medium in the role of special biological substances, and perform stem cell proliferation. After the determination of the function, the cells can be used as an in vitro normal cell experimental model.

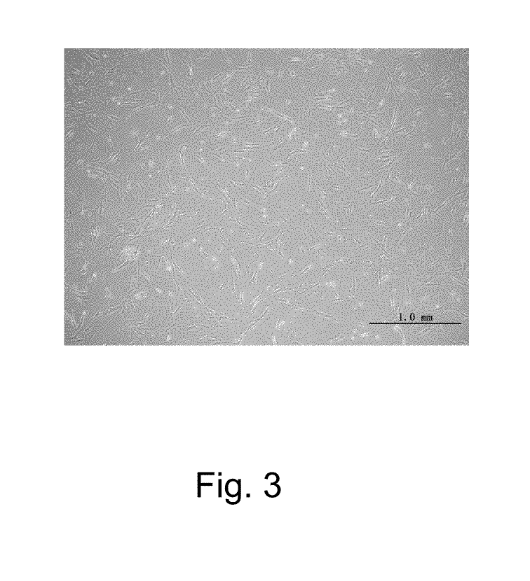

Referring to FIG. 3, it can be seen that the cells that are continuously proliferated begin to link into tissue in the culture medium, and the cells that have been linked into the tissue changes their shapes of cells from circular form to forms of the tissue cells, and part of the cells continue to proliferate. The tissue formed can be used as an in vitro tissue experimental model.

In the study of tissue cells used for tissue organs culture in vitro, the inventors have studied the source cells with proliferative ability which are culture at identical time and found that some cells began to divide into a single tissue cell, and soon became a terminal cell, no new tissues are formed; and the other part of the cells then continue to split and proliferate, and the proliferated cells continuously form tissues, several different forms of tissues together combines into a large tissue.

### Embodiment 2

Formation of Adipose Tissue, Induction and Differentiation of Bone and Cartilage

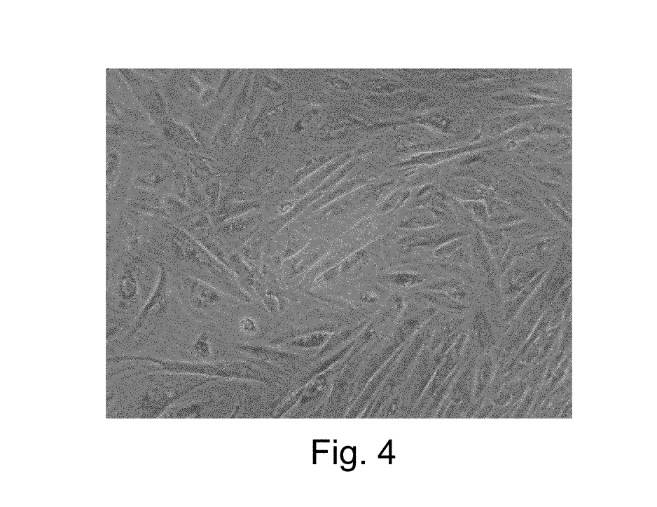

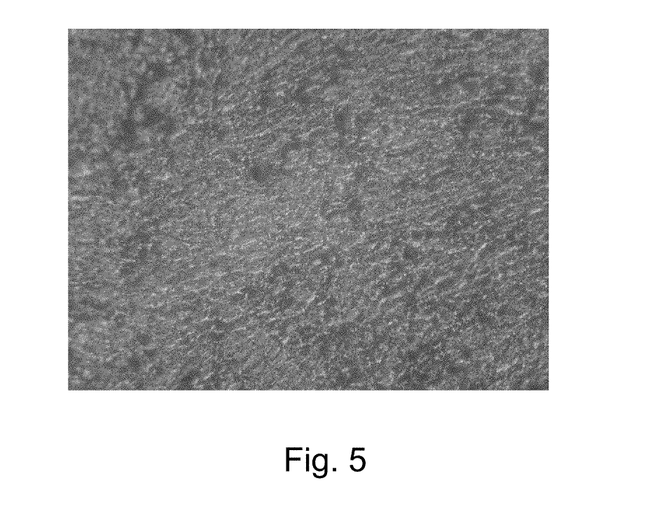

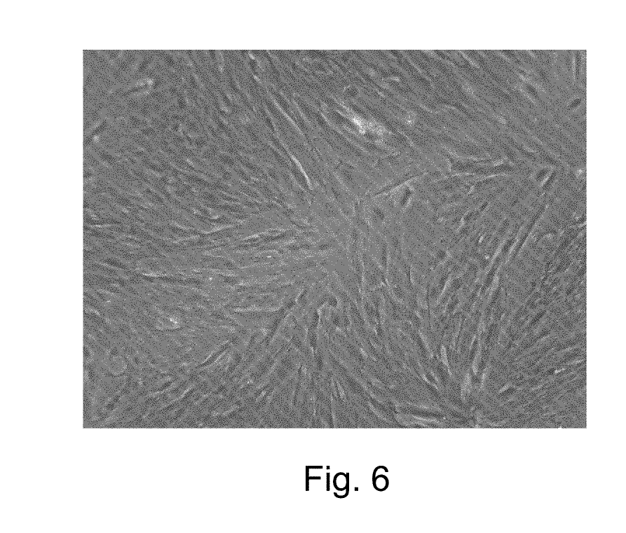

Referring to FIG. 3, continuously culture placental cells in the experimental group, continuously add three inducers of bone, cartilage and fat, two weeks later, adipose tissue, bone and cartilage are formed. See FIG. 4-5, respectively using different staining method, bone, cartilage, adipose tissue are formed under the microscope.

### Embodiment 3

Identification of Cell Surface Markers

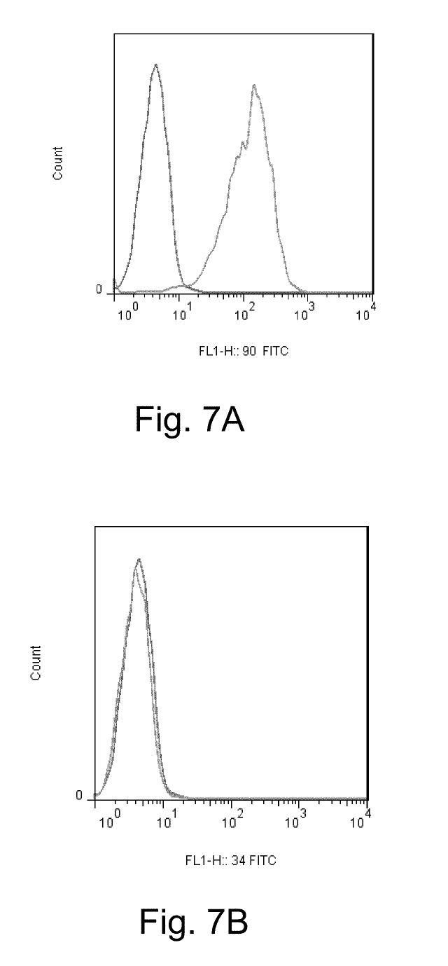

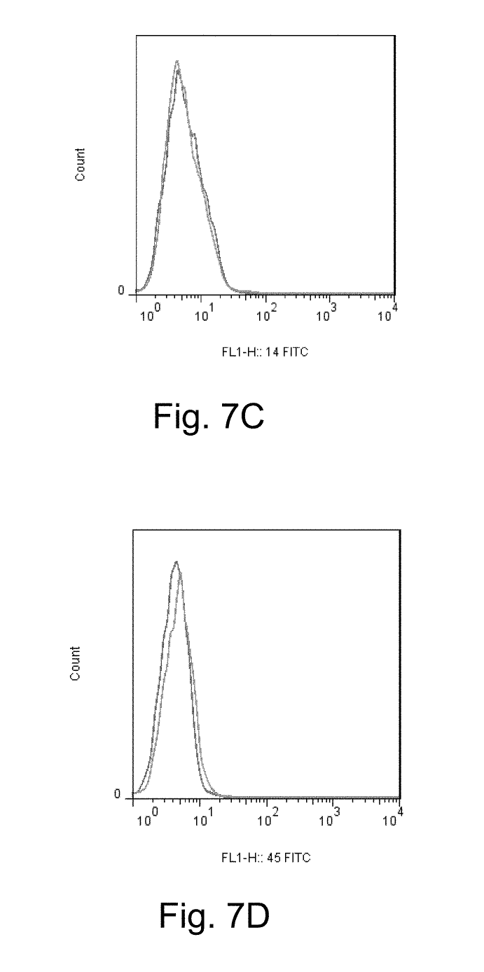

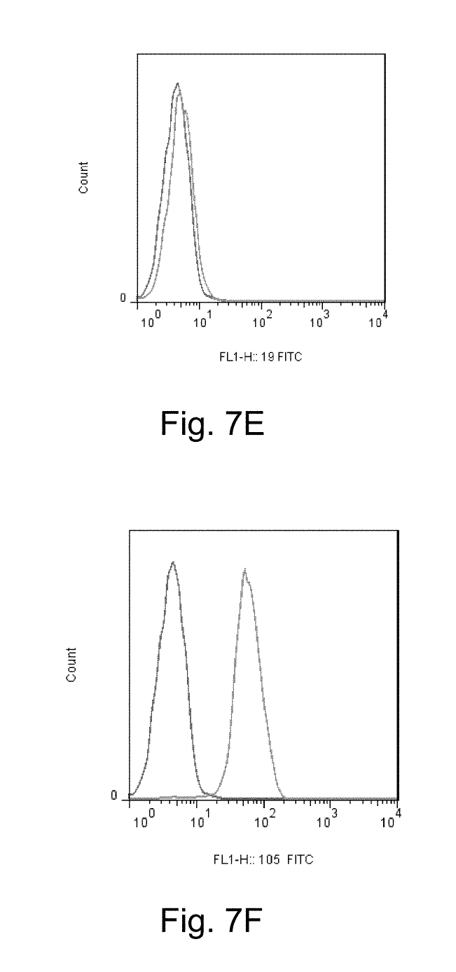

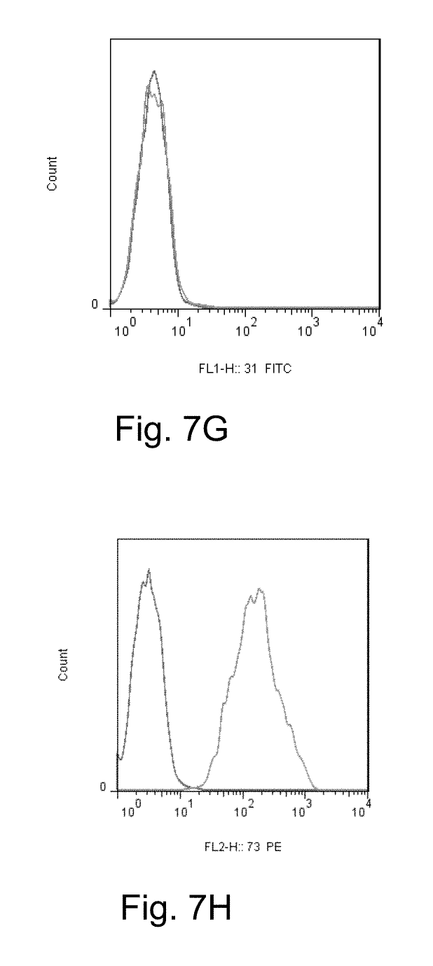

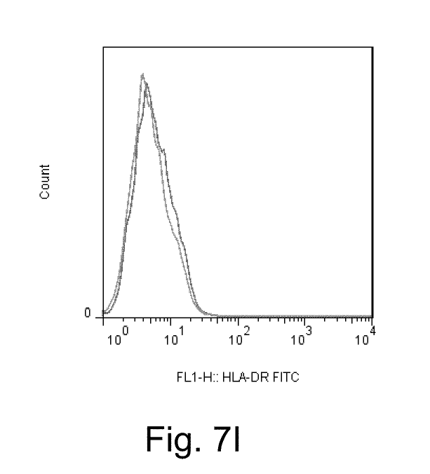

Referring to FIG. 7, the control group and the experimental group were set up in the same manner as in Example 1, and the substance of the present invention is not added to the control group. After 30 days of culture, cell surface markers were determined by flow cytometry. Referring to FIGS. 7A-7I, the experimental results are shown as below.

FIG. 7A shows CD90>95%, FIG. 7B shows CD34<2%; FIG. 7C shows CD14<2%, FIG. 7D shows CD45<2%, FIG. 7E shows CD19<2%, FIG. 7F shows CD105>95%, FIG. 7G shows CD31<2%, FIG. 7H shows CD73>95%, FIG. 7I shows HLA-DR<2%. The experimental group expresses CD90, CD73 and CD105 and CD45, CD14 and HLA-DR are not expressed. The control group do not express any of them.

### Embodiment 4

The origin of the placenta was replaced by a mouse, a hamster, a pig, or other animal, Embodiments 1-3 are repeated and the results are similar to those of Examples 1-3.

According to another preferred embodiment of the present invention, the components of the medium comprise:

1) 80% by mass fraction of amino acids selected from one or more member from the group consisting of: arginine, cystine, glutamine, histidine, isoleucine, leucine, lysine, methionine, phenylalanine, threonine, tryptophan and valine;

2) 4% by mass fraction of vitamins selected from one or more member from the group consisting of: biotin, choline, folic acid, nicotine, pantothenic acid, vitamin B6, vitamin B1 and riboflavin;

3) 0.4% by mass fraction of NaCl and 0.4% by mass fraction of KCl,

4) 5% by mass fraction of insulin and 5% by mass fraction of transferrin; and

5) 2.2% by mass fraction of glucose, 1.5% by mass fraction of penicillin and 1.5% by mass fraction of streptomycin.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method for culturing placental potential cell, comprising steps of:
(1) obtaining placental cells and/or tissue under aseptic condition;
(2) seeding the placental cells and/or the tissue in a culture medium for culturing, adding cell growth regulators to the culture medium, in such a manner that the placental potential cells grow to make the placental cells and/or the tissue into a proliferative state;
(3) culturing the placental potential cells to make the placental potential cells proliferate continuously into cells with characteristics of stem cells.

2. The method, as recited in claim 1, further comprises a step of: under in-vitro induction conditions, forming various types of tissues by cells which are proliferated.

3. The method, as recited in claim 1, wherein the inoculating the placental cells and/or the tissue in the culture medium for culturing of the step (2) comprises steps of:
(2-A) washing the placental cells and/or the tissue with phosphate buffer (PBS) containing at least two types of antibiotics comprising penicillin and streptomycin for at least 2 times, 30 seconds-3 minutes for each time;
(2-B) washing the placental cells and/or the tissue to a complete medium containing at least two types of antibiotics comprising the penicillin and the streptomycin for at least 2 times, 30 seconds-3 minutes for each time;
(2-C) cutting the placental cells and/or the tissue into explants with a size at a range of 1-5 mm³;
(2-D) placing the organ type explants into a center of the culture microplates of the culture plate in 1-5 mm interval, slightly pressing each of the organ type explants to make each of the organ type explants tightly cling to a surface of the culture plate;
(2-E) along each periphery of each of the culture microplates, adding 0.1-1.0 ml the complete medium to each of the culture microplates, preventing the complete medium from contacting with the organ type explants;
(2-F) sending the culture plate into an incubator at 37° C. with 1-10% CO₂ to pre-culture for 0.5-2 hours; and
(2-G) gently adding the complete medium to each of the microplates, preventing the explants from floating, and adding the cell growth regulator.

4. The method, as recited in claim 1, wherein the inoculating the placental cells and/or the tissue in the culture medium for culturing of the step (2) comprises steps of:
(2-a) in order to separate the placental tissue into individual cells, firstly putting the placental tissue into 4° C. pre-cooled phosphate buffered saline (PBS) containing at least two types of antibiotics comprising penicillin and streptomycin, washing for at least 3 times, 30 seconds to 3 minutes for each time; then cutting the placental tissue into minimal organ type explants with a size at a range of 1-5 mm³, washing for two times with cooled PBS containing at least two types of antibiotics; then sending the placental tissue into 0.1-0.5% neutral protease solution or 0.5-2% collagenase solution which is prepared by sterile PBS for digestion, wherein conditions are 36.5-37° C. constant temperature oscillation for 0.5-3 hours;
(2-b) gently mixing the explants by pipetting with a sampler or a straw; or pouring the explants digested with 0.1-0.5% dispase or 0.5-2% collagenase solution (in sterile PBS) onto a stainless steel sieve, grinding the explants with a syringe until the explants are completely discrete and become single cells; keeping mixture containing the single cells and digestive enzyme which is known to skills in the art for 2-8 minutes, discarding precipitated chunks and non-digestible connective tissue; transferring supernatant containing plenty of the single cells and the digestive enzyme to another centrifugal tube; filtering the supernatant once with a stainless steel filter, so as to obtain a mixture of the digestive enzyme and the single cell;
(2-c) centrifuging for 3-20 minutes with 1000-3000 r/min at 4° C., discarding supernatant containing the digestive enzyme, spinning the single cells by a vortex with PBS which is pre-cooled at 0.5-6° C.; then adding PBS pre-cooled at 0.5-6° C.; and mixing well;
(2-d) centrifuging for 3-20 minutes at 0.5-6° C. with a rate 1000-3000 r/min; discarding supernatant, adding cool PBS, oscillating with a vortex to spin the single cells; then adding PBS pre-cooled at 0.5-6° C.; mixing well; and counting the single cells;
(2-e) centrifuging for 3-20 minutes at 0.5-6° C. with a rate 1000-3000 r/min; discarding supernatant, adding alpha MEM culture solution containing 10-20% fetal bovine serum; oscillating by vortex to spin up the single cells, then adding the alpha MEM culture solution containing 10-20% fetal bovine serum to regulate the cell concentration at 1×10⁵/ml, mixing well to obtain a quantitative cell suspension;
(2-f) adding the cell suspension to a plastic porous plate which is a 96-well plate, 24-well plate, 12-well plate or a 6-well plate; wherein adding 200 μl to each well of the 96-well plate; adding 1 ml to each well of the 24-well plate; adding 2 mL to each well of the 12-well plate and adding 4 mL to each well of the 6-well plate;
(2-g) sending the plastic porous plate to a cell incubator containing 5% CO₂ at 36.5-37° C. for 12-48 hours until all the cells are adherent.

5. The method, as recited in claim 1, wherein a source of the placenta comprises: a spontaneous abortion human placenta, a mice placenta, a hamster placenta, a pig placenta or a cow placenta.

6. The method, as recited in claim 1, wherein components of the medium comprise:
1) amino acids selected from one or more member from the group consisting of: arginine, cystine, glutamine, histidine, isoleucine, leucine, lysine, methionine, phenylalanine, threonine, tryptophan and valine;
2)vitamins selected from one or more member from the group consisting of: biotin, choline, folic acid, nicotine, pantothenic acid, vitamin B6, vitamin B1 and riboflavin;
3)salt selected from one or more member from the group consisting of: NaCl, KCl, NaHPO₄, NaHCO₃, CaCl₂ and MgCl₂;
4)salt selected from one or more member from the group consisting of: insulin and transferrin;
5) 1 glucose, penicillin and streptomycin.

7. The method, as recited in claim 1, wherein the cell growth regulator is selected from the group consisting of: resveratrol, amino acids, fetal bovine serum and human serum.

8. The method, as recited in claim 1, wherein a content of the resveratrol in the medium is 10-60 ng/mL.

9. A placental potential cell prepared according to the method of claim 1.
